(19) United States
(12) Patent Application Publication
(10) Pub. No.: US 2023/0420907 A1
(43) Pub. Date: Dec. 28, 2023

(54) Laser Module and Methods Thereof

(71) Applicant: C. R. Bard, Inc., Franklin Lakes, NJ (US)

(72) Inventors: Paul Thomas Mannion, Eliot, ME (US); Jeff E. Franklin, Hamilton, OH (US); Tracey E. Knapp, Snellville, GA (US)

(21) Appl. No.: 18/036,347

(22) PCT Filed: Nov. 11, 2021
(86) PCT No.: PCT/US21/59003

§ 371 Date: May 10, 2023

Related U.S. Application Data
(60) Provisional application No. 63/116,021, filed on Nov. 19, 2020.

(51) Int. Cl. H01S 3/23; H01S 3/10; H01S 3/091

(52) U.S. Cl. CPC H01S 3/2383; H01S 3/10046; H01S 3/0912; H01S 3/161

(57) ABSTRACT

Disclosed are laser modules for laser systems and methods thereof that expand options for clinicians when using lasers in medical procedures such as holmium lasers in urological procedures. A laser module includes independently drivable laser-producing assemblies, laser optics, and a driver for driving the laser-producing assemblies. Each laser-producing assembly includes an optical resonator having a gain medium set among resonator optics for directing light through the gain medium for amplification of the light by stimulated emission. The laser optics combines two or more input laser beams produced by the laser-producing assemblies into a combined laser beam having a pulse energy, a pulse width, or a pulse repetition frequency resulting from a combination of the two-or-more input laser beams. The laser optics also directs at least a portion of the combined laser beam through an outlet of the laser module as an output laser beam.

This application claims the benefit of priority to U.S. Provisional Patent Application No. 63/116,021, filed Nov. 19, 2020, which is incorporated by reference in its entirety into this application.

### BACKGROUND

Advances in lasers and optical-fiber delivery systems have promoted the use of lasers in medical procedures. Indeed, lasers such as the holmium laser have been found useful for a variety of urological procedures including partial or full nephrectomies, laser-assisted trans-urethral resections of the prostate (“TURP”), treatment of tumors associated with superficial bladder cancer, or destruction of urinary stones. A number of settings including that for pulse energy, pulse frequency, and pulse width are available to clinicians on holmium laser systems for such urological procedures; however, the number of settings available on the foregoing holmium laser systems is currently limited, which, in turn, limits treatment options for the urological procedures. What is needed are laser modules for laser systems and methods thereof that expand options for clinicians when using holmium or other lasers in medical procedures.

Disclosed herein are laser modules and methods that address the foregoing.

### SUMMARY

Disclosed herein is a laser module including, in some embodiments, a plurality of independently drivable laser-producing assemblies, laser optics, and a printed circuit board assembly. Each laser-producing assembly of the plurality of laser-producing assemblies includes an optical resonator and a pump. The optical resonator includes a gain medium set among resonator optics configured to direct light through the gain medium for amplification of the light by stimulated emission. The pump is configured to pump energy into the gain medium to excite ions, atoms, or molecules of the gain medium for the stimulated emission. The laser optics is configured to independently combine two or more input laser beams produced by the plurality of laser-producing assemblies into an output laser beam having a pulse energy, a pulse width, or a pulse repetition frequency resulting from a combination of the two-or-more input laser beams. The laser optics is also configured to direct at least a portion of the output laser beam through an outlet of the laser module. The printed circuit board assembly includes a driver configured for independently driving each laser-producing assembly of the plurality of laser-producing assemblies with respect to at least a pulse energy, a pulse width, or a pulse repetition frequency of its input laser beam.

In some embodiments, the pulse repetition frequency of pulses of the output laser beam is double that of either of two input laser beams of the two-or-more input laser beams.

In some embodiments, pulses of a first input laser beam and pulses of a second input laser beam of the two input laser beams have a same pulse repetition interval. The pulses of the second input laser beam are delayed with respect to the pulses of the first input laser beam by half the pulse repetition interval.

In some embodiments, wherein the pulse repetition frequency of pulses of the output laser beam is quadruple that of any of four input laser beams of the two-or-more input laser beams.

In some embodiments, pulses of a first input laser beam, pulses of a second input laser beam, pulses of a third input laser beam, and pulses of a fourth input laser beam of four input laser beams have a same pulse repetition interval. The pulses of the second input laser beam are delayed with respect to the pulses of the first input laser beam by one-quarter the pulse repetition interval. The pulses of the third input laser beam are delayed with respect to the pulses of the first input laser beam by half the pulse repetition interval. The pulses of the fourth input laser beam are delayed with respect to the pulses of the first input laser beam by three-quarters the pulse repetition interval.

In some embodiments, pulses of the output laser beam are tuples of pulses of the two-or-more input laser beams.

In some embodiments, pulses of a first input laser beam and pulses of a second input laser beam of the two-or-more input laser beams have a same pulse repetition interval. The pulses of the second input laser beam are delayed with respect to the pulses of the first input laser beam by at least a pulse width of the pulses of the first input laser beam plus no more than the pulse width of the pulses of the first input laser beam.

In some embodiments, the pulse energy of pulses of the output laser beam is about double that of either of two input laser beams of the two-or-more input laser beams for half the pulses of the output laser beam.

In some embodiments, pulses of a first input laser beam of the two input laser beams have a pulse repetition interval half that of pulses of a second input laser beam of the two input laser beams. Every other pulse of the pulses of the first input laser beam temporally coincide with a pulse of the pulses of the second input laser beam.

In some embodiments, each input laser beam of the two-or-more input laser beams has about a same pulse energy and about a same pulse width.

In some embodiments, the output laser beam is a continuous wave of pulses of two input laser beams of the two-or-more input laser beams.

In some embodiments, pulses of a first input laser beam and pulses of a second input laser beam of the two input laser beams have a same pulse width and a same pulse repetition interval. The pulses of the second input laser beam are delayed with respect to the pulses of the first input laser beam by the pulse width of the first and second input laser beams.

In some embodiments, pulses of a first input laser beam and pulses of a second input laser beam of the two input laser beams have a different pulse width and a same pulse repetition interval. The pulses of the second input laser beam are delayed with respect to the pulses of the first input laser beam by a pulse width of the first input laser beam.

In some embodiments, the pulse width of the first input laser beam is half that of the second input laser beam.

In some embodiments, the pulse energy of the output laser beam is modulated. The first input laser beam or the second input laser beam has a greater pulse energy than the second input laser beam or the first input laser beam, respectively.

In some embodiments, the output laser beam is a continuous wave of a first input laser beam and a pulsed wave of a second input laser beam. The peak power of the output laser beam is modulated in accordance with a pulse repetition interval of the second input laser beam.

Also disclosed herein is a method of a laser module for a medical system. The method includes, in some embodiments, an input laser-driving step, an input laser-combining step, and an output laser-directing step. The input laser-driving step includes independently driving with a driver of a printed circuit board assembly each laser-producing assembly of a plurality of laser-producing assemblies with respect to at least a pulse energy, a pulse width, or a pulse repetition frequency of its input laser beam. The input laser-driving step includes an energy-pumping step. The energy-pumping step includes pumping energy into a gain medium with a pump to excite ions, atoms, or molecules of the gain medium for amplification of light by stimulated emission. The input laser-combining step includes independently combining with laser optics two or more input laser beams produced by the plurality of laser-producing assemblies into an output laser beam having a pulse energy, a pulse width, or a pulse repetition frequency resulting from a combination of the two-or-more input laser beams. The output laser-directing step includes directing at least a portion of the output laser beam through an outlet of the laser module.

In some embodiments, the pulse repetition frequency of pulses of the output laser beam is double that of either of two input laser beams of the two-or-more input laser beams after combining the two input laser beams with the laser optics in the input laser-combining step. Pulses of each input laser beam of the two input laser beams have a same pulse repetition interval.

In some embodiments, the input laser-driving step includes delaying pulses of a second input laser beam of the two input laser beams with respect to pulses of a first input laser beam of the two input laser beams by half the pulse repetition interval shared by the two input laser beams.

In some embodiments, the pulse repetition frequency of pulses of the output laser beam is quadruple that of any of four input laser beams of the two-or-more input laser beams after combining the four input laser beams with the laser optics in the input laser-combining step. Pulses of each input laser beam of the four input laser beams have a same pulse repetition interval.

In some embodiments, the input laser-driving step includes delaying pulses of a second input laser beam of the four input laser beams with respect to pulses of a first input laser beam of the four input laser beams by one-quarter the pulse repetition interval shared by the four input laser beams. The input laser-driving step also includes delaying pulses of a third input laser beam of the four input laser beams with respect to the pulses of the first input laser beam by half the pulse repetition interval shared by the four input laser beams. The input laser-driving step also includes delaying pulses of a fourth input laser beam of the four input laser beams with respect to the pulses of the first input laser beam by three-quarters the pulse repetition interval shared by the four input laser beams.

In some embodiments, pulses of the output laser beam are tuples of pulses of the two-or-more input laser beams after the input laser-combining step. Pulses of each input laser beam of the two-or-more input laser beams have a same pulse repetition interval.

In some embodiments, the input laser-driving step includes delaying pulses of a second input laser beam of the two-or-more input laser beams with respect to pulses of a first input laser beam of the two-or-more input laser beams by at least a pulse width of the pulses of the first input laser beam plus no more than the pulse width of the pulses of the first input laser beam.

In some embodiments, the pulse energy of pulses of the output laser beam is about double that of either of two input laser beams of the two-or-more input laser beams for half the pulses of the output laser beam after the input laser-combining step.

In some embodiments, input laser-driving step includes pulsing a first input laser beam of the two input laser beams with a pulse repetition interval half that of a second input laser beam of the two input laser beams. Every other pulse of the first input laser beam temporally coincides with a pulse of the second input laser beam.

In some embodiments, the input laser-driving step includes generating each input laser beam of the two-or-more input laser beams with about a same pulse energy and about a same pulse width.

In some embodiments, the input laser-driving step includes pulsing two input laser beams of the two-or-more input laser beams to generate the output beam as a continuous wave.

In some embodiments, the pulsing of the two input laser beams includes pulsing a first input laser beam and a second input laser beam of the two input laser beams with a same pulse width and a same pulse repetition interval while delaying pulses of the second input laser beam with respect to pulses of the first input laser beam by the pulse width of the first and second input laser beams.

In some embodiments, the pulsing of the two input laser beams includes pulsing a first input laser beam and a second input laser beam of the two input laser beams with a different pulse width and a same pulse repetition interval while delaying pulses of the second input laser beam with respect to pulses of the first input laser beam by a pulse width of the first input laser beam.

In some embodiments, the pulse width of the first input laser beam is half that of the second input laser beam.

In some embodiments, the pulsing of the first input laser beam and the second input laser beam includes pulsing the first input laser beam or the second input laser beam with a greater pulse energy than the second input laser beam or the first input laser beam, respectively, thereby generating the output laser beam with a modulated pulse energy.

In some embodiments, the input laser-driving step includes generating a continuous wave of a first input laser beam of the two-or-more input laser beams and pulsing a second input laser beam of the two-or-more input laser beams, thereby generating the output laser beam with a modulated peak power in accordance with a pulse repetition interval of the second input laser beam.

These and other features of the concepts provided herein will become more apparent to those of skill in the art in view of the accompanying drawings and following description, which describe particular embodiments of such concepts in greater detail.

### DRAWINGS

FIG. 1 illustrates a laser module in accordance with some embodiments.

FIG. 2 illustrates a laser-producing assembly of the laser module in accordance with some embodiments.

FIG. 3 illustrates another laser-producing assembly of the laser module in accordance with some embodiments.

FIG. 4 illustrates another laser-producing assembly of the laser module in accordance with some embodiments.

FIG. 5 illustrates a block diagram for electronics of the laser module in accordance with some embodiments.

FIG. 6 provides a profile of an output laser beam resulting from a combination of two input laser beams in accordance with some embodiments.

FIG. 7 provides a profile of an output laser beam resulting from a combination of four input laser beams in accordance with some embodiments.

FIG. 8 provides a profile of an output laser beam resulting from another combination of two input laser beams in accordance with some embodiments.

FIG. 9 provides a profile of an output laser beam resulting from another combination of two input laser beams in accordance with some embodiments.

FIG. 10 provides a profile of an output laser beam resulting from another combination of two input laser beams in accordance with some embodiments.

FIG. 11 provides a profile of an output laser beam resulting from another combination of two input laser beams in accordance with some embodiments.

FIG. 12 provides a profile of an output laser beam resulting from another combination of two input laser beams in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

As used herein, a pulse repetition interval (“PRI”) is a time interval between two adjacent pulses of a laser beam. A pulse repetition frequency (“PRF”) is a rate of the pulses of the laser beam per unit time. The pulse repetition interval and the pulse repetition frequency are inversely related.

Unless defined otherwise, all technical and scientific terms used herein have the same meaning as commonly understood by those of ordinary skill in the art.

As set forth above, advances in lasers and optical-fiber delivery systems have promoted the use of lasers in medical procedures such as the holmium laser in a variety of urological procedures. A number of settings including that for pulse energy, pulse frequency, and pulse width are available to clinicians on holmium laser systems for the urological procedures; however, the number of settings available on such holmium laser systems is currently limited, which, in turn, limits treatment options for the urological procedures. What is needed are laser modules for laser systems and methods thereof that expand options for clinicians when using holmium or other lasers in medical procedures.

Disclosed herein are laser modules and methods that address the foregoing.

### Laser Modules

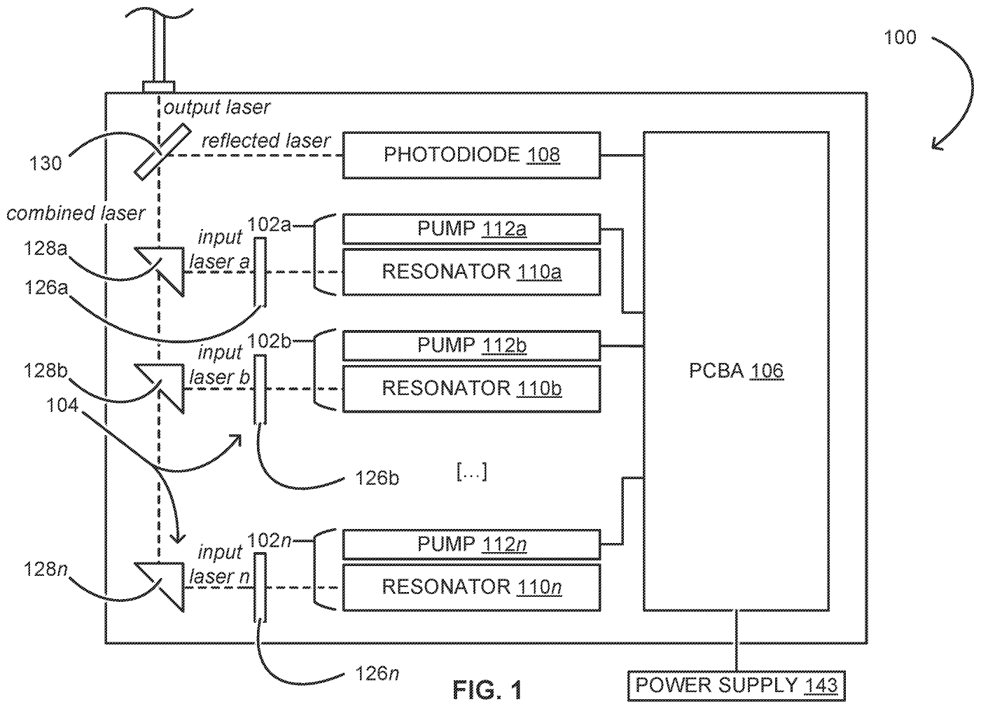

FIG. 1 illustrates a laser module 100 in accordance with some embodiments.

As shown, the laser module 100 includes a plurality of independently drivable laser-producing assemblies 102a, 102b, . . . , 102n, which are collectively referred to herein as laser-producing assemblies 102, laser optics 104, and a printed circuit board assembly (“PCBA”) 106. The laser module 100 can further include a photodetector such as a photodiode 108 or a photodetector array such as a photodiode array with the laser optics 104 configured therefor.

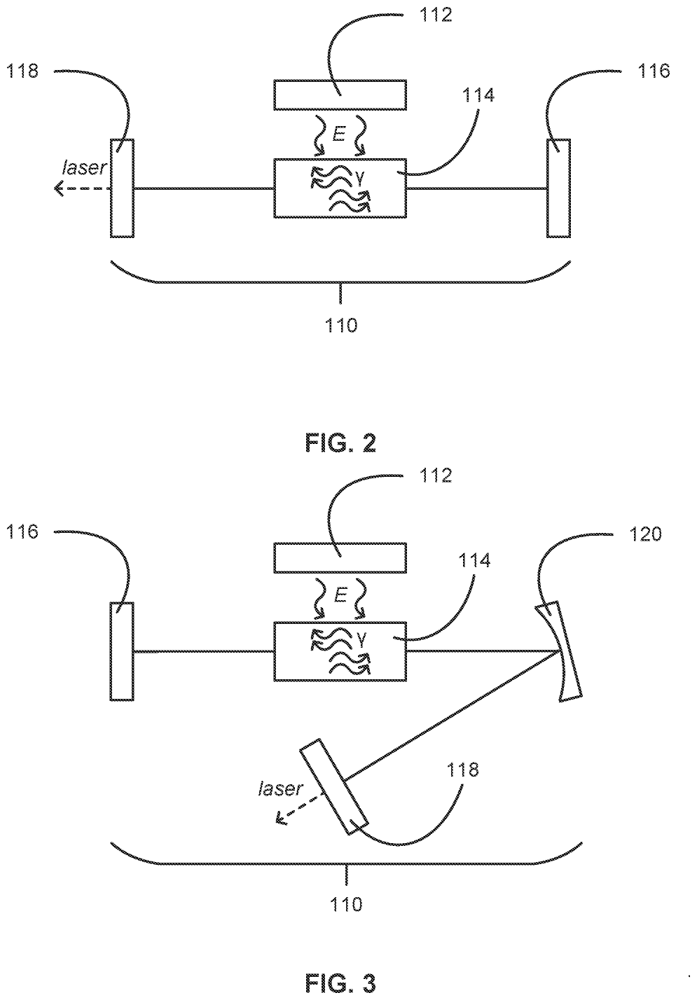

FIGS. 2-4 illustrate the plurality of laser-producing assemblies 102 of the laser module 100 in accordance with some embodiments.

Each laser-producing assembly 102a, 102b, . . . , 102n of the plurality of laser-producing assemblies 102 respectively includes an optical resonator 110 (e.g., an optical resonator 110a, an optical resonator 110b, . . . , an optical resonator 110n paired with a pump 112 (e.g., a pump 112a, a pump 112b, . . . , a pump 112n).

The optical resonator 110 includes a gain medium 114 set among resonator optics configured to direct light through the gain medium 114 for amplification of the light by stimulated emission of photons γ. The gain medium 114 can include a crystal including a rare-earth metal ion-doped yttrium aluminum garnet (“YAG”) crystal such as holmium-doped YAG (“Ho:YAG”), neodymium-doped YAG (“Nd:YAG”), ytterbium-doped YAG (“Yb:YAG”) or erbium-doped YAG (“Er:YAG”); a ceramic including a rare-earth metal ion-doped YAG ceramic such as neodymium-doped YAG; a glass including a rare-earth metal ion-doped phosphate or silicate glass; a semiconductor such as gallium arsenide, indium gallium arsenide, or gallium nitride; a liquid solution including one or more laser-active organic molecules such as a dye; or a gas including one or more laser-active atoms or molecules such as carbon dioxide.

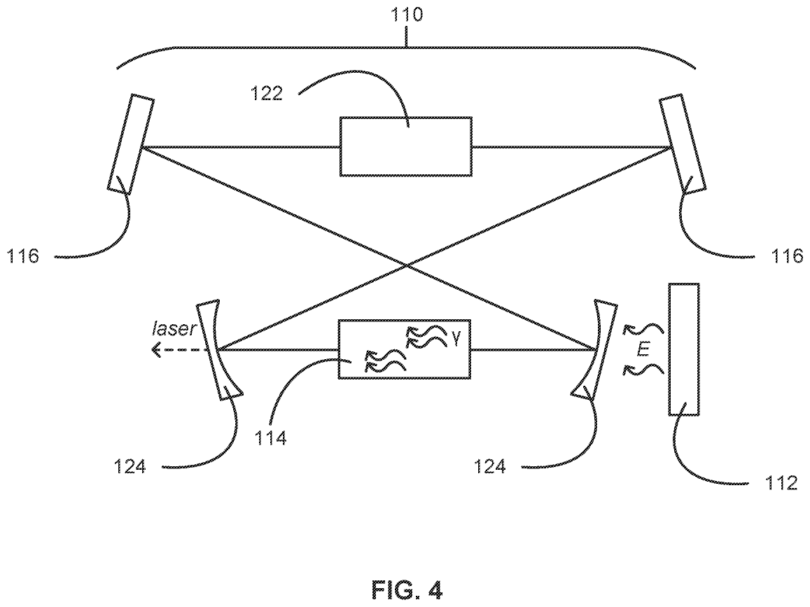

The resonator optics can include any number or type of optical elements including laser mirrors, Faraday isolators, or the like needed to effectuate a linear resonator or a ring resonator for the optical resonator 110. The laser mirrors can include dielectric mirrors, dichroic mirrors, or the like configured as highly reflective or partially transmissive plane or curved mirrors as needed for the linear or ring resonator. For example, the optical resonator 110 of FIG. 2 is a side-pumped linear resonator including a highly reflective plane mirror 116 and a partially transmissive output coupler 118. In another example, the optical resonator 110 of FIG. 3 is a side-pumped linear resonator including the highly reflective plane mirror 116, a highly reflective curved mirror 120, and the partially transmissive output coupler 118. In another example, the optical resonator 110 of FIG. 4 is a ring resonator including a Faraday isolator 122, which transmits light in a first direction while blocking light in a second, opposing direction, two instances of the highly reflective plane mirror 116, and two instances of a partially transmissive curved mirror 124. Each instance of the two instances of the partially transmissive curved mirror 124 can be configured for a passing a different wavelength of light therethrough. Indeed, one instance of the two instances of the partially transmissive curved mirror 124 can function as the output coupler.

The pump 112 is configured to pump energy E into the gain medium 114 to excite ions, atoms, or molecules of the gain medium 114 for the stimulated emission of photons γ. The pump 112 can be configured to optically or electrically pump the gain medium 114 from a side or an end of the gain medium 114 as in FIGS. 2 and 3, respectively. When the gain medium 114 is a crystal, ceramic, or glass, for example, the pump 112 can be a laser diode or a discharge lamp. In another example, when the gain medium 114 is a semiconductor, the pump 112 can be an electrical pump.

The laser optics 104 is configured to independently combine two or more input laser beams (e.g., laser beam a, laser beam b, . . . , laser beam n of FIG. 1) produced by the plurality of laser-producing assemblies 102 into a combined laser beam having a pulse energy, pulse repetition frequency, or pulse width resulting from a combination of the two-or-more input laser beams. For example, the laser optics 104 can include a collimating lens 126 (e.g., a collimating lens 126a, a collimating lens 126b, . . . , a collimating lens 126n,) and a prism 128 (e.g., a prism 128a, a prism 128b, . . . , a prims 128n,) for each laser-producing assembly 102a, 102b, . . . , 102n of the plurality of laser-producing assemblies 102 for combining the two-or-more input laser beams into the combined laser beam. The laser optics 104 is also configured to direct a portion up to an entirety of the combined laser beam through an outlet of the laser module 100 as an output laser beam. It should be understood the output laser beam referred to herein is the combined laser beam albeit with an optional portion directed to a photodetector such as the photodiode 108.

When the photodiode 108 is present, the laser optics 104 can further include a beam splitter 130 or the like configured to direct a portion of the combined laser beam to the photodiode 108 as a reflected laser beam. When a photodiode array is present, the laser optics 104 can further include a plurality of beam splitters or the like configured to direct a portion of each input laser beam to the photodiode array as a reflected input laser beam.

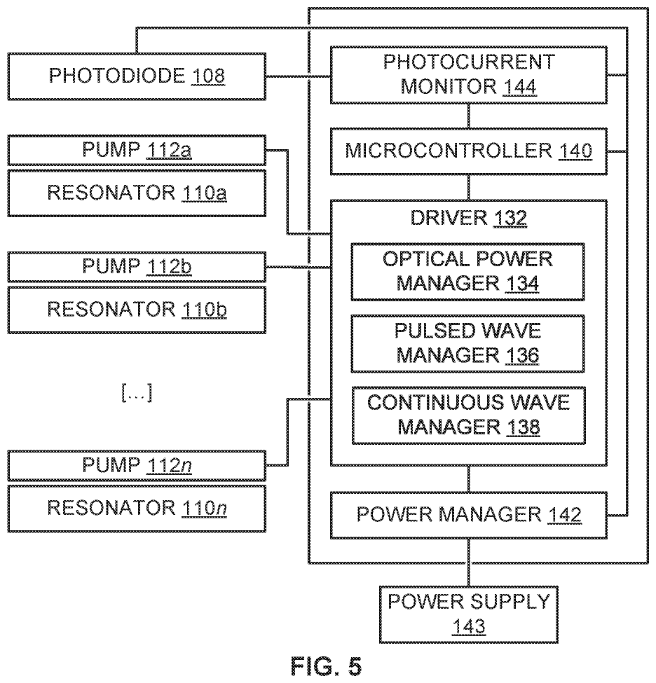

FIG. 5 illustrates a block diagram for electronics of the laser module in accordance with some embodiments.

The PCBA 106 includes a driver 132 configured for independently driving each laser-producing assembly 102a, 102b, . . . , 102n of the plurality of laser-producing assemblies 102 with respect to at least a pulse energy, a pulse repetition frequency, or a pulse width of its input laser beam. To effectuate such driving, the driver 132 supplies an appropriate drive current to each laser-producing assembly 102a, 102b, . . . , 102n or the pump 112 thereof in accordance with an optical power manager 134 configured to control the power or pulse power and a pulsed wave manager 136 configured to control both the pulse width and the pulse repetition frequency. On account of, for example, pulse energy being the product of a pulse power over a period of time for a pulse, the optical power manager 134 and the pulsed wave manager 136, together, are configured to control pulse energy. In addition, the driver 132 can be configured for independently driving each laser-producing assembly 102a, 102b, . . . , 102n of the plurality of laser-producing assemblies 102 with respect to continuous-wave operation of its input laser beam. To effectuate such driving, the driver 132 can supply an appropriate drive current to each laser-producing assembly 102a, 102b, . . . , 102n or the pump 112 thereof in accordance with a continuous wave manager 138 configured to drive the continuous-wave operation.

The PCBA 106 also includes a microcontroller 140 and a power manager 142 coupled to a power supply 143. The microcontroller 140 includes one or more central processing units (“CPUs”), program memory having executable instructions, and at least a small amount of random-access memory (“RAM”) configured to control the laser module 100. The power manager 142 is configured to manage power distribution and consumption for the laser module 100, thereby maintaining a cooler operating temperature for the laser module 100.

When the photodiode 108 is present, the PCBA 106 also includes a photocurrent monitor 144 configure to monitor an instant photocurrent produced by the photodiode 108 in accordance with the reflected laser beam, which instant photocurrent is proportional to the reflected laser beam, and which reflected laser beam, in turn, is proportional to the output laser beam per the beam splitter 130 or the like. Comparison of the instant photocurrent of the reflected laser beam with an expected photocurrent for the reflected laser beam by photocurrent-comparison logic of the microcontroller 140 enables the driver 132 to cooperatively and independently adjust the driving of any one or more laser-producing assemblies of the plurality of laser-producing assemblies 102. For example, if the output laser beam is expected to have a profile like that provided in FIG. 6 but comparison of the instant photocurrent of the reflected laser beam to the expected photocurrent for the reflected laser beam indicates delayed pulses corresponding to that of input laser b have a diminished pulse energy, the driver 132 is configured to cooperatively adjust the driving of the laser-producing assembly 102b.

When a photodiode array is present, the PCBA 106 also includes the photocurrent monitor 144 but configured to monitor parallel instant photocurrents produced by the photodiode array in accordance with reflected input laser beams. Comparison of the instant photocurrents of the reflected input laser beams with expected photocurrents for the reflected input laser beams by the photocurrent-comparison logic of the microcontroller 140 enables the driver 132 to cooperatively and independently adjust the driving of any one or more laser-producing assemblies of the plurality of laser-producing assemblies 102. For example, if a reflected input laser beam (e.g., a reflected portion of input laser b), as a counterpart to that forming the output laser beam, is expected to contribute to the profile of the output laser beam like that provided in FIG. 6 but comparison of the instant photocurrent of the reflected input laser beam to the expected photocurrent for the reflected input laser beam indicates pulses have a diminished pulse energy, the driver 132 is configured to cooperatively and independently adjust the driving of the laser-producing assembly therefor.

### Output Laser Beam Profiles

As set forth above, the laser optics 104 is configured to independently combine the two-or-more input laser beams (e.g., laser beam a, laser beam b, . . . , laser beam n of FIG. 1) produced by the plurality of laser-producing assemblies 102 into the combined laser beam having a pulse energy, pulse repetition frequency, or pulse width resulting from the combination of the two-or-more input laser beams. FIGS. 6-12 provide various profiles of the output laser beam resulting from the combination of the two-or-more input laser beams, each of which profiles provide an additional option for effectuating a medical procedure with the output laser beam.

FIG. 6 provides a profile of an output laser beam resulting from a combination of two input laser beams (e.g., input laser a and input laser b) of the two-or-more input laser beams in accordance with some embodiments.

As shown, the pulse repetition frequency of pulses of the output laser beam is double that of either laser beam of the two input laser beams combined to form the output laser beam. Indeed, pulses of a first input laser beam (e.g., input laser a) and pulses of a second input laser beam (e.g., input laser b) of the two input laser beams have a same pulse repetition interval, but the pulses of the second input laser beam are delayed with respect to the pulses of the first input laser beam by half the pulse repetition interval in the output laser beam in order to double the pulse repetition frequency in the output laser beam. For the pulses of the first input laser beam in the output laser beam, the pulses occur at t₀+n PRI for n= [symbol] ₀, where the pulse repetition interval is equal to t₂-t₀ for an initial time point t₀ and a time point t₂ after an intervening pulse of the second input laser beam. For the pulses of the second input laser beam in the output laser beam, the pulses occur at (t₀+D)+n PRI for n= [symbol] ₀, where the delay (“D”) is equal to ½ PRI. In a numerical example, if the pulses of both the first input laser beam and the second input laser beam are every 0.45 ms for a pulse repetition frequency of 2200 Hz, but the pulses of the second input laser beam are delayed 0.225 ms from the pulses of the first input laser beam, then the pulse repetition frequency of the output laser beam is effectively 4400 Hz.

While the pulse widths PW₁ and PW₂ of the first input laser beam and the second input laser beam, respectively, are equal (e.g., 0.05 ms) in the profile of the output laser beam shown in FIG. 6, the pulse widths need not be equal. In addition, while the pulse powers P₁ and P₂ of the first input laser beam and the second input laser beam, respectively, are equal in the profile of the output laser beam shown in FIG. 6, the pulse powers need not be equal. Indeed, any combination of pulse widths and pulse powers are possible.

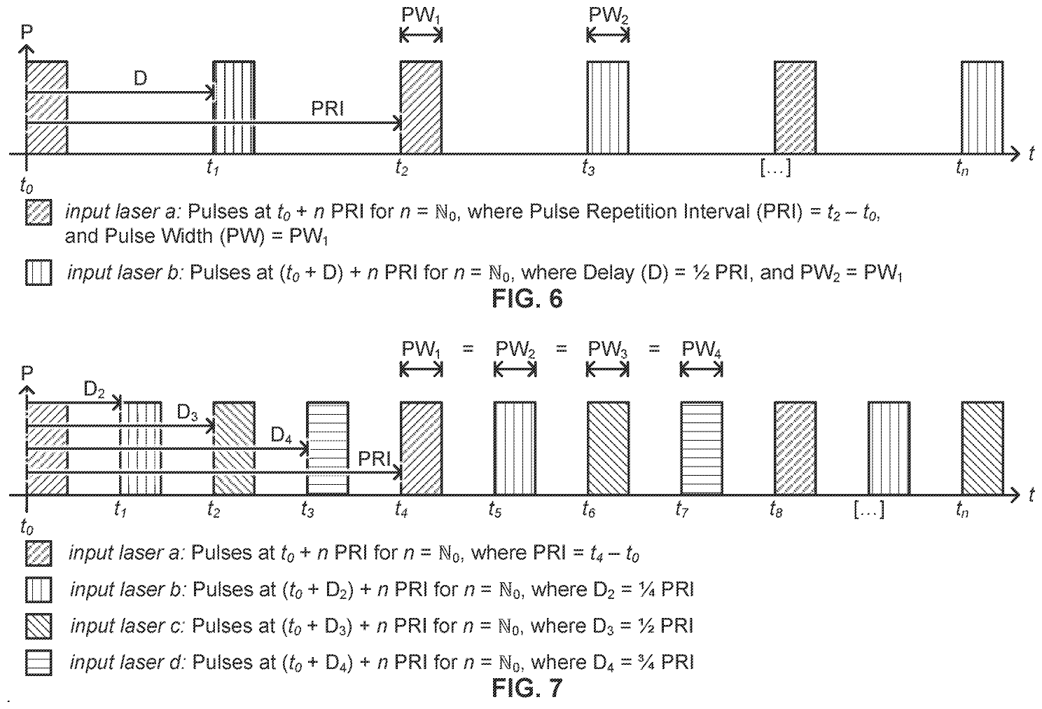

FIG. 7 provides a profile of an output laser beam resulting from a combination of four input laser beams (e.g., input laser a, input laser b, input laser c, and input laser d) of the two-or-more input laser beams in accordance with some embodiments.

As shown, the pulse repetition frequency of pulses of the output laser beam is quadruple that of any laser beam of the four input laser beams combined to form the output laser beam. Indeed, pulses of a first input laser beam (e.g., input laser a), pulses of a second input laser beam (e.g., input laser b), pulses of a third input laser beam (e.g., input laser c), and pulses of a fourth input laser beam (e.g., input laser d) of the four input laser beams have a same pulse repetition interval, but the pulses of the second input laser beam are delayed with respect to the pulses of the first input laser beam by one-quarter the pulse repetition interval, the pulses of the third input laser beam are delayed with respect to the pulses of the first input laser beam by half the pulse repetition interval, and the pulses of the fourth input laser beam are delayed with respect to the pulses of the first input laser beam by three-quarters the pulse repetition interval in the output laser beam in order to quadruple the pulse repetition frequency in the output laser beam. For the pulses of the first input laser beam in the output laser beam, the pulses occur at t₀+n PRI for n= [symbol] ₀, where the pulse repetition interval is equal to t₄-t₀ for the initial time point t₀ and a time point t₄ after intervening pulses of the second, third, and fourth input laser beams. For the pulses of the second input laser beam in the output laser beam, the pulses occur at (t₀+D₂)+n PRI for n= [symbol] ₀, where the delay (“D₂”) is equal to ¼ PRI. For the pulses of the third input laser beam in the output laser beam, the pulses occur at (t₀+D₃)+n PRI for n= [symbol] ₀, where the delay (“D₃”) is equal to ½ PRI. For the pulses of the fourth input laser beam in the output laser beam, the pulses occur at (t₀+D₄)+n PRI for n= [symbol] ₀, where the delay (“D₄”) is equal to ¾ PRI. In a numerical example, if the pulses of each input laser beam of the first, second, third, and fourth input laser beams are every 0.45 ms for a pulse repetition frequency of 2200 Hz, but the pulses of the second, third, and fourth input laser beams are successively delayed 0.1125 ms from each other starting from the pulses of the first input laser beam, then the pulse repetition frequency of the output laser beam is effectively 8800 Hz.

While the pulse widths PW₁, PW₂, PW₃, and PW₄ of the first, second, third, and fourth input laser beams, respectively, are equal (e.g., 0.05 ms) in the profile of the output laser beam shown in FIG. 7, the pulse widths need not be equal. In addition, while the pulse powers P₁, P₂, P₃, and P₄ of the first, second, third, and fourth input laser beams, respectively, are equal in the profile of the output laser beam shown in FIG. 7, the pulse powers need not be equal. Indeed, any combination of pulse widths and pulse powers are possible.

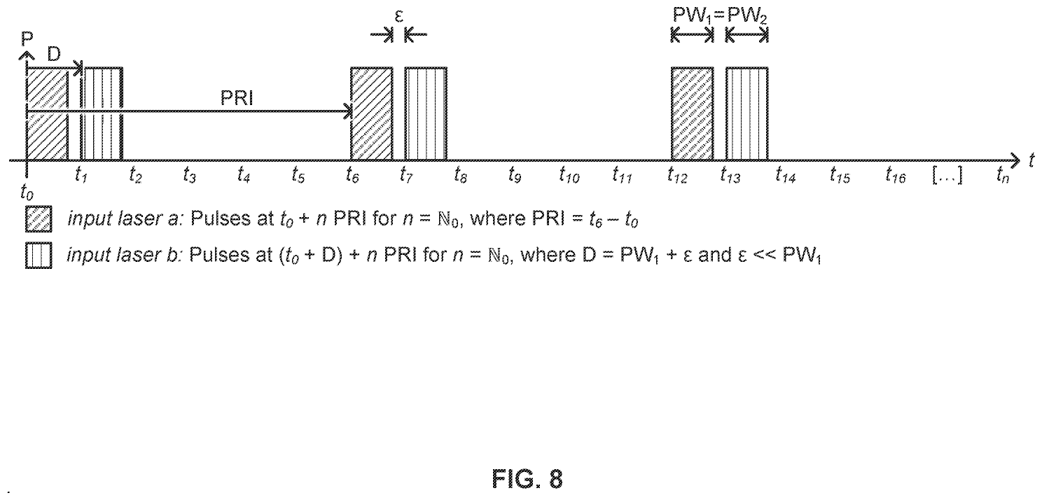

FIG. 8 provides a profile of an output laser beam resulting from another combination of two input laser beams (e.g., input laser a and input laser b) of the two-or-more input laser beams in accordance with some embodiments.

As shown, pulses of the output laser beam are couples of pulses of the two input laser beams combined to form pulse bursts in the output laser beam; however, it should be understood the pulses of the output laser beam being the couples of the pulses of the two input laser beams is but one example of the pulses of the output laser beam being tuples of pulses of the two-or-more input laser beams. Indeed, the pulses of the output laser beam can alternatively be triples of pulses of three input laser beams combined to form the output laser beam, quadruples of pulses of four input laser beams combined to form the output laser beam, and so on. As to the pulses of the output laser beam being the couples of the pulses of the two input laser beams, pulses of a first input laser beam (e.g., input laser a) and pulses of a second input laser beam (e.g., input laser b) of the two input laser beams have a same pulse repetition interval, but the pulses of the second input laser beam are delayed with respect to the pulses of the first input laser beam by at least a pulse width of the pulses of the first input laser beam plus no more than the pulse width of the pulses of the first input laser beam in order to form the pulse bursts in the output laser beam. Notably, the pulses of the second input laser beam delayed with respect to the pulses of the first input laser beam by at least the pulse width of the pulses of the first input laser beam plus no more than a small fraction of the pulse width of the pulses of the first input laser beam provide a tighter coupling of the pulses of the first and second input laser beams. For the pulses of the first input laser beam in the output laser beam, the pulses occur at t₀+n PRI for n= [symbol] ₀, where the pulse repetition interval is equal to t₆-t₀ for the initial time point t₀ and a time point t₆ after an intervening pulse of the second input laser beam plus an amount of additional time. For the pulses of the second input laser beam in the output laser beam, the pulses occur at (t₀+D)+n PRI for n= [symbol] ₀, where the delay is equal to PW₁ or PW₁+ε for ε«PW₁. In a numerical example, if the pulses of both the first input laser beam and the second input laser beam have a pulse width (“PW”) of 0.05 ms and occur every 0.45 ms for a pulse repetition frequency of 2200 Hz, but the pulses of the second input laser beam are delayed 0.06 ms from the pulses of the first input laser beam, then the couples of the pulses of the two input laser beams effectively form pulse bursts in the output laser beam with a frequency of 2200 Hz.

While the pulse widths PW₁ and PW₂ of the first input laser beam and the second input laser beam, respectively, are equal (e.g., 0.05 ms) in the profile of the output laser beam shown in FIG. 8, the pulse widths need not be equal. In addition, while the pulse powers P₁ and P₂ of the first input laser beam and the second input laser beam, respectively, are equal in the profile of the output laser beam shown in FIG. 8, the pulse powers need not be equal. Indeed, any combination of pulse widths and pulse powers are possible.

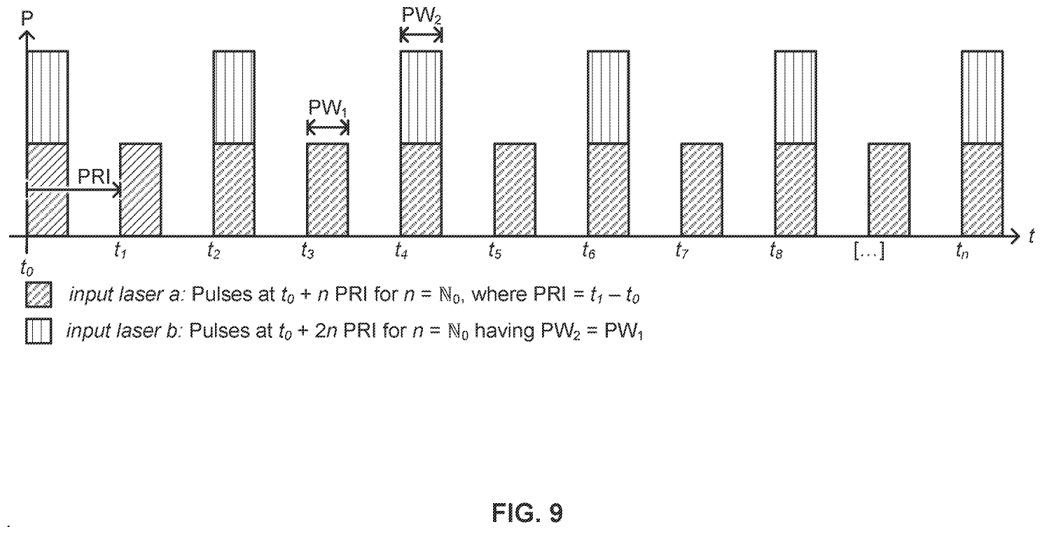

FIG. 9 provides a profile of an output laser beam resulting from another combination of two input laser beams (e.g., input laser a and input laser b) of the two-or-more input laser beams in accordance with some embodiments.

As shown, the pulse energy of pulses of the output laser beam is about double that of either laser beam of the two input laser beams combined to form the output laser beam for half the pulses of the output laser beam. In order to form such a modulated output laser beam (e.g., a power-modulated output laser), pulses of a first input laser beam (e.g., input laser a) of the two input laser beams have a pulse repetition interval half that of pulses of a second input laser beam (e.g., input laser b) of the two input laser beams such that every other pulse of the pulses of the first input laser beam temporally coincides with a pulse of the pulses of the second input laser beam, optionally subsequent to a delay in the pulses of either the first or second input laser beam, wherein the delay is a multiple of the pulse repetition interval. For the pulses of the first input laser beam in the output laser beam, the pulses occur at t₀+n PRI for n= [symbol] ₀, where the pulse repetition interval is equal to t₁-t₀ for the initial time point t₀ and a time point t₁ for an adjacent pulse of the first input laser beam. For the pulses of the second input laser beam in the output laser beam, the pulses occur at t₀+2n PRI for n = [symbol] ₀. Again, the pulses of either the first or second input laser beam can be delayed by a multiple of the pulse repetition interval. In such cases, the first term (i.e., t₀) in either t₀+n PRI for the first input laser beam or the t₀+2n PRI for the second input laser beam is replaced by t₀+D, where the delay is equal to m PRI for m= [symbol] ₀. In a numerical example, if the pulses of the first input laser beam are every 0.45 ms for a pulse repetition frequency of 2200 Hz, the pulses of the second input laser beam are every 0.90 ms for a pulse repetition frequency of 1100 Hz, and the pulses of the second input laser beam temporally coincide with the pulses of the first input laser beam, then the pulse energy of pulses of the output laser beam is about double that of either laser beam of the two input laser beams for half the pulses of the output laser beam (i.e., those having the pulse repetition frequency of 1100 Hz).

While the pulse widths PW₁ and PW₂ of the first input laser beam and the second input laser beam, respectively, are equal (e.g., 0.05 ms) in the profile of the output laser beam shown in FIG. 9, the pulse widths need not be equal. In addition, while the pulse powers P₁ and P₂ of the first input laser beam and the second input laser beam, respectively, are equal in the profile of the output laser beam shown in FIG. 9, the pulse powers need not be equal. Indeed, any combination of pulse widths and pulse powers are possible. In addition, any number of input laser beams of the two-or-more input laser beams can be combined to form the output laser beam.

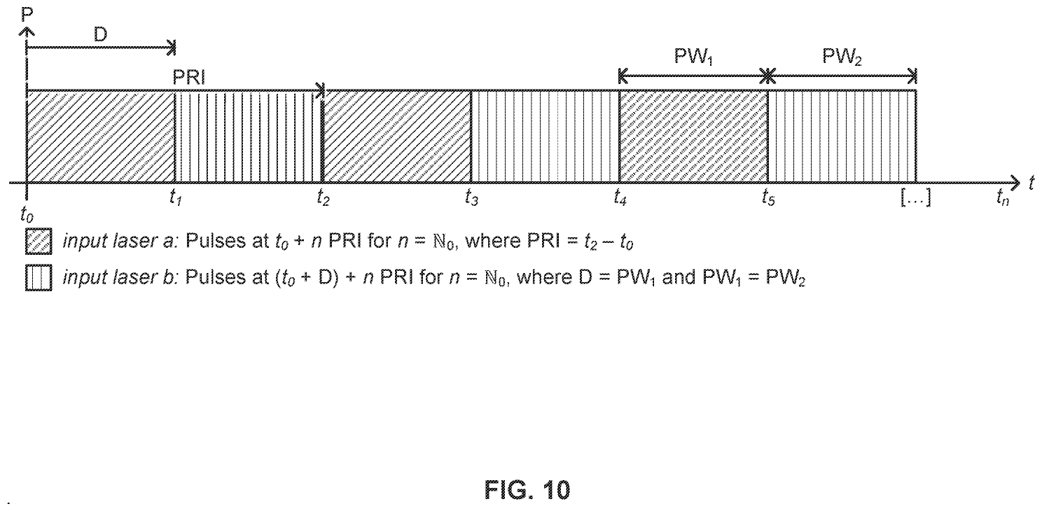

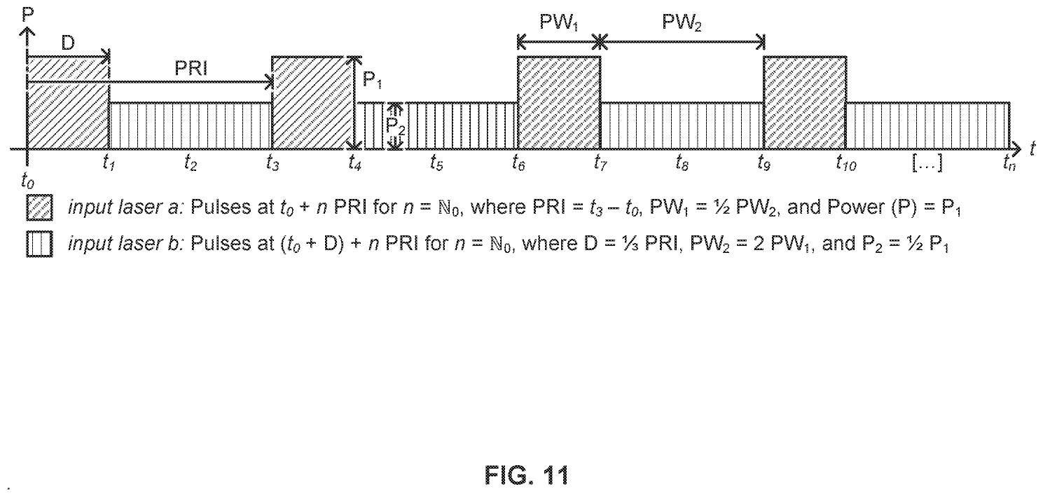

FIG. 10 provides a profile of an output laser beam resulting from another combination of two input laser beams (e.g., input laser a and input laser b) of the two-or-more input laser beams in accordance with some embodiments.

As shown, the output laser beam is an unmodulated continuous wave of pulses of the two input laser beams combined to form the output laser beam. Indeed, pulses of a first input laser beam (e.g., input laser a) and pulses of a second input laser beam (e.g., input laser b) of the two input laser beams have a same pulse energy, a same pulse width, and a same pulse repetition interval, but the pulses of the second input laser beam are delayed with respect to the pulses of the first input laser beam by the pulse width of the first and second input laser beams in order to form the unmodulated continuous wave the output laser beam. For the pulses of the first input laser beam in the output laser beam, the pulses occur at t₀+n PRI for n= [symbol] ₀, where the pulse repetition interval is equal to t₂-t₀ for the initial time point t₀ and a time point t₂ after an intervening pulse of the second input laser beam. For the pulses of the second input laser beam in the output laser beam, the pulses occur at (t₀+D)+n PRI for n= [symbol] ₀, where the delay is equal to the pulse width (“PW₁”) of the pulses of the first input laser beam, which, in turn, is equal to the pulse width (“PW₂”) of the pulses of the second input laser beam. In a numerical example, if the pulses of both the first input laser beam and the second input laser beam have a pulse width of 0.225 ms and occur every are every 0.45 ms, but the pulses of the second input laser beam are delayed 0.225 ms from the pulses of the first input laser beam, then the output laser beam is effectively a continuous wave.

While the pulse widths PW₁ and PW₂ of the first input laser beam and the second input laser beam, respectively, are equal (e.g., 0.225 ms) in the profile of the output laser beam shown in FIG. 10, the pulse widths need not be equal. In addition, while the pulse powers P₁ and P₂ of the first input laser beam and the second input laser beam, respectively, are equal in the profile of the output laser beam shown in FIG. 10, the pulse powers need not be equal. Indeed, as set forth below, FIG. 11 provides a modulated continuous wave for an output laser beam formed from two input laser beams having pulses of different pulse powers and pulse widths.

FIG. 11 provides a profile of an output laser beam resulting from another combination of two input laser beams (e.g., input laser a and input laser b) of the two-or-more input laser beams in accordance with some embodiments.

As shown, the output laser beam is a modulated continuous wave (e.g., a power-modulated continuous wave) of pulses of the two input laser beams combined to form the output laser beam. Indeed, pulses of a first input laser beam (e.g., input laser a) and pulses of a second input laser beam (e.g., input laser b) of the two input laser beams have a different pulse energy, a different pulse width, and a same pulse repetition interval, but the pulses of the second input laser beam are delayed with respect to the pulses of the first input laser beam by a pulse width of the first input laser beam, which is half that of the second input laser beam, in order to form the modulated continuous wave of the output laser beam. In addition, the pulses of the first input laser beam have double the pulse energy of the pulses of the second input laser beam. For the pulses of the first input laser beam in the output laser beam, the pulses occur at t₀+n PRI for n= [symbol] ₀, where the pulse repetition interval is equal to t₃-t₀ for the initial time point t₀ and a time point t₃ after an intervening pulse of the second input laser beam. For the pulses of the second input laser beam in the output laser beam, the pulses occur at (t₀+D)+n PRI for n= [symbol] ₀ with a pulse power (“P₂”) equal to half a pulse power (“P₁”) of the pulses of the first input laser beam, where the delay is equal to the pulse width of the pulses of the first input laser beam, which, in turn, is half the pulse width of the pulses of the second input laser beam. In a numerical example, if the pulses of the first input laser beam have a pulse energy double that of the pulses of the second input laser beam with a pulse width of 0.225 ms and occur every 0.675 ms for a pulse repetition frequency of 4400 Hz, but the pulses of the second input laser beam have a pulse width of 0.45 ms and are delayed 0.225 ms from the pulses of the first input laser beam, then the output laser beam is effectively a modulated continuous wave with pulses of doubled pulse energy a pulse repetition frequency of 4400 Hz.

While the pulse width PW₁ of the first input laser beam is half that of the second input laser beam in the profile of the output laser beam shown in FIG. 10, the pulse widths can be in some other relationship such as equal or opposite with the pulse width PW₂ of the second input laser beam half that of the first input laser beam. In addition, while the pulse power P₁ of the first input laser beam is double that of the second input laser beam in the profile of the output laser beam shown in FIG. 10, the pulse powers can be in some other relationship such as opposite with the pulse power P₂ of the second input laser beam double that of the first input laser beam.

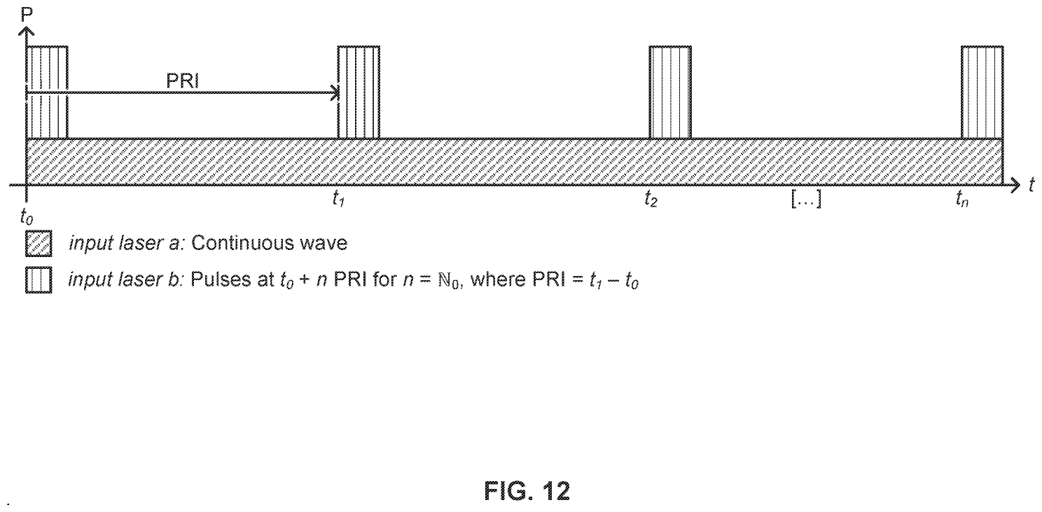

FIG. 12 provides a profile of an output laser beam resulting from another combination of two input laser beams (e.g., input laser a and input laser b) of the two-or-more input laser beams in accordance with some embodiments.

As shown, the output laser beam is a modulated continuous wave (e.g., a power-modulated continuous wave) of the two input laser beams combined to form the output laser beam. Indeed, a continuous wave of a first input laser beam (e.g., input laser a) of the two input laser beams is combined with pulses of a second input laser beam (e.g., input laser b) of the two input laser beams in order to form the modulated continuous wave of the output laser beam. The pulses of the second input laser beam have a pulse repetition frequency by which the pulse energy modulates in the output laser beam. For the pulses of the second input laser beam in the output laser beam, the pulses occur at t₀+n PRI for n= [symbol] ₀, where the pulse repetition interval is equal to t₁-t₀ for the initial time point t₀ and a time point t₁ for an adjacent pulse of the second input laser beam. In a numerical example, if the pulses of the second input laser beam are every 0.45 ms for a pulse repetition frequency of 2200 Hz, then the pulse energy in the output laser beam also modulates with a frequency of 2200 Hz.

While the power P₁ of the first input laser beam is half that of the pulse power P₂ of the second input laser beam, the power P₁ and the pulse power P₂ can be in some other relationship. Indeed, any combination of pulse widths and pulse powers are possible.

### Methods

Methods of the laser module 100 include methods of using the laser module 100 in a medical system. For example, a method of using the laser module includes an input laser-driving step, an input laser-combining step, and an output laser-directing step.

The input laser-driving step includes independently driving with the driver 132 of the PCBA 106 each laser-producing assembly 102a, 102b, . . . , 102n of the plurality of laser-producing assemblies 102 with respect to at least a pulse energy, a pulse repetition frequency, or a pulse width of its input laser beam.

The input laser-driving step includes an energy-pumping step. The energy-pumping step includes pumping energy into the gain medium 114 with the pump 112 to excite ions, atoms, or molecules of the gain medium 114 for amplification of light by stimulated emission.

The input laser-combining step includes independently combining with the laser optics 104 the two-or-more input laser beams produced by the plurality of laser-producing assemblies 102 into the output laser beam having a pulse energy, pulse repetition frequency, or pulse width resulting from a combination of the two-or-more input laser beams.

The output laser-directing step includes directing at least a portion of the output laser beam through an outlet of the laser module 100.

Additional details for each step of the input laser-driving step and the input laser-combining step is set forth below with respect to FIGS. 6-12, which provide various profiles of the output laser beam resulting from the combination of the two-or-more input laser beams.

Again, FIG. 6 provides a profile of an output laser beam resulting from a combination of two input laser beams (e.g., input laser a and input laser b) of the two-or-more input laser beams in accordance with some embodiments.

In view of FIG. 6, the input laser-driving step can include delaying pulses of a second input laser beam (e.g., input laser b) of the two input laser beams with respect to pulses of a first input laser beam (e.g., input laser a) of the two input laser beams by half the pulse repetition interval shared by the two input laser beams. After combining the two input laser beams with the laser optics 104 in the input laser-combining step, the pulse repetition frequency of pulses of the output laser beam is double that of either of two input laser beams.

Again, FIG. 7 provides a profile of an output laser beam resulting from a combination of four input laser beams (e.g., input laser a, input laser b, input laser c, and input laser d) of the two-or-more input laser beams in accordance with some embodiments.

In view of FIG. 7, the input laser-driving step includes delaying pulses of a second input laser beam (e.g., input laser b) of the four input laser beams with respect to pulses of a first input laser beam (e.g., input laser a) of the four input laser beams by one-quarter the pulse repetition interval shared by the four input laser beams. The input laser-driving step also includes delaying pulses of a third input laser beam (e.g., input laser c) of the four input laser beams with respect to the pulses of the first input laser beam by half the pulse repetition interval shared by the four input laser beams. The input laser-driving step also includes delaying pulses of a fourth input laser beam (e.g., input laser d) of the four input laser beams with respect to the pulses of the first input laser beam by three-quarters the pulse repetition interval shared by the four input laser beams. After combining the four input laser beams with the laser optics 104 in the input laser-combining step, the pulse repetition frequency of pulses of the output laser beam is quadruple that of any of four input laser beams.

Again, FIG. 8 provides a profile of an output laser beam resulting from another combination of two input laser beams (e.g., input laser a and input laser b) of the two-or-more input laser beams in accordance with some embodiments.

In view of FIG. 8, the input laser-driving step includes delaying pulses of a second input laser beam (e.g., input laser b) of the two input laser beams with respect to pulses of a first input laser beam (e.g., input laser a) of the two input laser beams by at least a pulse width of the pulses of the first input laser beam plus no more than the pulse width of the pulses of the first input laser beam, wherein pulses of each input laser beam of the two input laser beams have a same pulse repetition interval. After combining the two input laser beams with the laser optics 104 in the input laser-combining step, pulses of the output laser beam are tuples (e.g., doubles) of pulses of the two input laser beams.

Again, FIG. 9 provides a profile of an output laser beam resulting from another combination of two input laser beams (e.g., input laser a and input laser b) of the two-or-more input laser beams in accordance with some embodiments.

In view of FIG. 9, the input laser-driving step includes pulsing a first input laser beam (e.g., input laser a) of the two input laser beams with a pulse repetition interval half that of a second input laser beam (e.g., input laser b) of the two input laser beams. Optionally, the input laser-driving step also includes generating each input laser beam of the two input laser beams with about a same pulse energy and about a same pulse width. Regardless, every other pulse of the first input laser beam temporally coincides with a pulse of the second input laser beam such that the pulse energy of pulses of the output laser beam is about double that of the two input laser beams for half the pulses of the output laser beam after the input laser-combining step.

Again, FIGS. 10 and 11 provide profiles of output laser beams resulting from two other combinations of two input laser beams (e.g., input laser a and input laser b) of the two-or-more input laser beams in accordance with some embodiments.

In view of FIGS. 10 and 11, the input laser-driving step includes pulsing the two input laser beams to generate the output beam as a continuous wave. The pulsing of the two input laser beams includes pulsing a first input laser beam (e.g., input laser a) and a second input laser beam (e.g., input laser a) of the two input laser beams with a same pulse width (see FIG. 10) or a different pulse width (see FIG. 11) and a same pulse repetition interval. In view of FIG. 10, the pulsing of the two input laser beams also includes delaying pulses of the second input laser beam with respect to pulses of the first input laser beam by the pulse width of the first and second input laser beams. In view of FIG. 11, however, the pulsing of the two input laser beams includes delaying the pulses of the second input laser beam with respect to the pulses of the first input laser beam by a pulse width of the first input laser beam, which is half that of the second input laser beam. Further in view of FIG. 11, the pulsing of the first input laser beam and the second input laser beam also includes pulsing the first input laser beam with a greater pulse energy than the second input laser beam, thereby generating the output laser beam with a modulated pulse energy.

Again, FIG. 12 provides a profile of an output laser beam resulting from another combination of two input laser beams (e.g., input laser a and input laser b) of the two-or-more input laser beams in accordance with some embodiments.

In view of FIG. 12, the input laser-driving step includes generating a continuous wave of a first input laser beam (e.g., input laser a) of the two input laser beams and pulsing a second input laser beam (e.g., input laser a) of the two input laser beams, thereby generating the output laser beam with a modulated peak power in accordance with a pulse repetition interval of the second input laser beam.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A laser module for a medical system, comprising:
a plurality of independently drivable laser-producing assemblies, each laser-producing assembly thereof including:
an optical resonator including a gain medium set among resonator optics configured to direct light through the gain medium for amplification of the light by stimulated emission; and
a pump configured to pump energy into the gain medium to excite ions, atoms, or molecules of the gain medium for the stimulated emission;
laser optics configured to independently combine two or more input laser beams produced by the plurality of laser-producing assemblies into an output laser beam and direct at least a portion of the output laser beam through an outlet of the laser module, the output laser beam having a pulse energy, a pulse width, or a pulse repetition frequency resulting from a combination of the two-or-more input laser beams; and
a printed circuit board assembly including a driver configured for independently driving each laser-producing assembly of the plurality of laser-producing assemblies with respect to at least a pulse energy, a pulse width, or a pulse repetition frequency of its input laser beam.

2. The laser module of claim 1, wherein the pulse repetition frequency of pulses of the output laser beam is double that of either of two input laser beams of the two-or-more input laser beams.

3. The laser module of claim 2, wherein pulses of a first input laser beam and pulses of a second input laser beam of the two input laser beams have a same pulse repetition interval, the pulses of the second input laser beam delayed with respect to the pulses of the first input laser beam by half the pulse repetition interval.

4. The laser module of claim 1, wherein the pulse repetition frequency of pulses of the output laser beam is quadruple that of any of four input laser beams of the two-or-more input laser beams.

5. The laser module of claim 4, wherein pulses of a first input laser beam, pulses of a second input laser beam, pulses of a third input laser beam, and pulses of a fourth input laser beam of four input laser beams have a same pulse repetition interval, the pulses of the second input laser beam delayed with respect to the pulses of the first input laser beam by one-quarter the pulse repetition interval, the pulses of the third input laser beam delayed with respect to the pulses of the first input laser beam by half the pulse repetition interval, and the pulses of the fourth input laser beam delayed with respect to the pulses of the first input laser beam by three-quarters the pulse repetition interval.

6. The laser module of claim 1, wherein pulses of the output laser beam are tuples of pulses of the two-or-more input laser beams.

7. The laser module of claim 6, wherein pulses of a first input laser beam and pulses of a second input laser beam of the two-or-more input laser beams have a same pulse repetition interval, the pulses of the second input laser beam delayed with respect to the pulses of the first input laser beam by at least a pulse width of the pulses of the first input laser beam plus no more than the pulse width of the pulses of the first input laser beam.

8. The laser module of claim 1, wherein the pulse energy of pulses of the output laser beam is about double that of either of two input laser beams of the two-or-more input laser beams for half the pulses of the output laser beam.

9. The laser module of claim 8, wherein pulses of a first input laser beam of the two input laser beams have a pulse repetition interval half that of pulses of a second input laser beam of the two input laser beams, every other pulse of the pulses of the first input laser beam temporally coinciding with a pulse of the pulses of the second input laser beam.

10. The laser module of claim 2, wherein each input laser beam of the two-or-more input laser beams has about a same pulse energy and about a same pulse width.

11. The laser module of claim 1, wherein the output laser beam is a continuous wave of pulses of two input laser beams of the two-or-more input laser beams.

12. The laser module of claim 11, wherein pulses of a first input laser beam and pulses of a second input laser beam of the two input laser beams have a same pulse width and a same pulse repetition interval, the pulses of the second input laser beam delayed with respect to the pulses of the first input laser beam by the pulse width of the first and second input laser beams.

13. The laser module of claim 11, wherein pulses of a first input laser beam and pulses of a second input laser beam of the two input laser beams have a different pulse width and a same pulse repetition interval, the pulses of the second input laser beam delayed with respect to the pulses of the first input laser beam by a pulse width of the first input laser beam.

14. The laser module of claim 13, wherein the pulse width of the first input laser beam is half that of the second input laser beam.

15. The laser module of claim 12, wherein the pulse energy of the output laser beam is modulated, the first input laser beam or the second input laser beam having a greater pulse energy than the second input laser beam or the first input laser beam, respectively.

16. The laser module of claim 1, wherein the output laser beam is a continuous wave of a first input laser beam and a pulsed wave of a second input laser beam, the peak power of the output laser beam modulated in accordance with a pulse repetition interval of the second input laser beam.

17. A method of a laser module for a medical system, comprising:
independently driving with a driver of a printed circuit board assembly each laser-producing assembly of a plurality of laser-producing assemblies with respect to at least a pulse energy, a pulse width, or a pulse repetition frequency of its input laser beam, the driving including pumping energy into a gain medium with a pump to excite ions, atoms, or molecules of the gain medium for amplification of light by stimulated emission;
independently combining with laser optics two or more input laser beams produced by the plurality of laser-producing assemblies into an output laser beam having a pulse energy, a pulse width, or a pulse repetition frequency resulting from a combination of the two-or-more input laser beams; and
directing at least a portion of the output laser beam through an outlet of the laser module.

18. The method of claim 17, wherein the pulse repetition frequency of pulses of the output laser beam is double that of either of two input laser beams of the two-or-more input laser beams after combining the two input laser beams with the laser optics, pulses of each input laser beam of the two input laser beams having a same pulse repetition interval.

19. The method of claim 18, wherein the driving of each laser-producing assembly of the plurality of laser-producing assemblies includes delaying pulses of a second input laser beam of the two input laser beams with respect to pulses of a first input laser beam of the two input laser beams by half the pulse repetition interval shared by the two input laser beams.

20. The method of claim 17, wherein the pulse repetition frequency of pulses of the output laser beam is quadruple that of any of four input laser beams of the two-or-more input laser beams after combining the four input laser beams with the laser optics, pulses of each input laser beam of the four input laser beams having a same pulse repetition interval.

21. The method of claim 20, wherein the driving of each laser-producing assembly of the plurality of laser-producing assemblies includes delaying pulses of a second input laser beam of the four input laser beams with respect to pulses of a first input laser beam of the four input laser beams by one-quarter the pulse repetition interval shared by the four input laser beams, pulses of a third input laser beam of the four input laser beams with respect to the pulses of the first input laser beam by half the pulse repetition interval shared by the four input laser beams, and pulses of a fourth input laser beam of the four input laser beams with respect to the pulses of the first input laser beam by three-quarters the pulse repetition interval shared by the four input laser beams.

22. The method of claim 17, wherein pulses of the output laser beam are tuples of pulses of the two-or-more input laser beams after the combining of the two-or-more input laser beams with the laser optics, pulses of each input laser beam of the two-or-more input laser beams having a same pulse repetition interval.

23. The method of claim 22, wherein the driving of each laser-producing assembly of the plurality of laser-producing assemblies includes delaying pulses of a second input laser beam of the two-or-more input laser beams with respect to pulses of a first input laser beam of the two-or-more input laser beams by at least a pulse width of the pulses of the first input laser beam plus no more than the pulse width of the pulses of the first input laser beam.

24. The method of claim 17, wherein the pulse energy of pulses of the output laser beam is about double that of either of two input laser beams of the two-or-more input laser beams for half the pulses of the output laser beam after the combining of the two-or-more input laser beams with the laser optics.

25. The method of claim 24, wherein the driving of each laser-producing assembly of the plurality of laser-producing assemblies includes pulsing a first input laser beam of the two input laser beams with a pulse repetition interval half that of a second input laser beam of the two input laser beams, every other pulse of the first input laser beam temporally coinciding with a pulse of the second input laser beam.

26. The method of claim 18, wherein the driving of each laser-producing assembly of the plurality of laser-producing assemblies includes generating each input laser beam of the two-or-more input laser beams with about a same pulse energy and about a same pulse width.

27. The method of claim 17, wherein the driving of each laser-producing assembly of the plurality of laser-producing assemblies includes pulsing two input laser beams of the two-or-more input laser beams to generate the output beam as a continuous wave.

28. The method of claim 27, wherein the pulsing of the two input laser beams includes pulsing a first input laser beam and a second input laser beam of the two input laser beams with a same pulse width and a same pulse repetition interval while delaying pulses of the second input laser beam with respect to pulses of the first input laser beam by the pulse width of the first and second input laser beams.

29. The method of claim 27, wherein the pulsing of the two input laser beams includes pulsing a first input laser beam and a second input laser beam of the two input laser beams with a different pulse width and a same pulse repetition interval while delaying pulses of the second input laser beam with respect to pulses of the first input laser beam by a pulse width of the first input laser beam.

30. The method of claim 29, wherein the pulse width of the first input laser beam is half that of the second input laser beam.

31. The method of claim 28, wherein the pulsing of the first input laser beam and the second input laser beam includes pulsing the first input laser beam or the second input laser beam with a greater pulse energy than the second input laser beam or the first input laser beam, respectively, thereby generating the output laser beam with a modulated pulse energy.

32. The method of claim 17, wherein the driving of each laser-producing assembly of the plurality of laser-producing assemblies includes generating a continuous wave of a first input laser beam of the two-or-more input laser beams and pulsing a second input laser beam of the two-or-more input laser beams, thereby generating the output laser beam with a modulated peak power in accordance with a pulse repetition interval of the second input laser beam.
